(19) United States
(12) Plant Patent Application Publication
(10) Pub. No.: US 2015/0282403 P1
(43) Pub. Date: Oct. 1, 2015

(54) Medinilla hybrid plant named 'ROYAL PEARLZ'

(50) ;

(71) Applicants: Elly Bak, Assendelft (NL); Nicolas Steur, Assendelft (NL)

(72) Inventors: Elly Bak, Assendelft (NL); Nicolas Steur, Assendelft (NL)

(21) Appl. No.: 13/999,857

(22) Filed: Mar. 28, 2014

(51) Int. Cl. A01H 5/00

(52) U.S. Cl. PLT/226

(57) ABSTRACT

A new and distinct Medinilla sp. plant named ‘ROYAL PEARLZ’, characterized by an erect shrub plant, measuring about 30 cm in total height (when first flowering) and about 45-70 cm in diameter; green colored leaves measuring about 15-20 cm in length and about 10-20 cm in width; pendulous panicle, measuring about 36 cm in length and about 15 cm in diameter, with red-purple (RHS 72C) flowers.

### LATIN NAME OF THE GENUS AND SPECIES

Medinilla hybrid

### VARIETY DENOMINATION

‘ROYAL PEARLZ’

### BACKGROUND OF THE INVENTION

The present invention relates to a new and distinct hybrid cultivar of Medinilla plant, botanically known as medinilla sp., hereinafter referred to by the cultivar name ‘ROYAL PEARLZ’. The genus of Medinilla belongs to the Melastimataceae family.

The new Medinilla ‘ROYAL PEARLZ’ is a product of a planned breeding program conducted by the inventor, Ely Bak and Nico D. M. Steur, in Assendelft, The Netherlands. The objective of the breeding program is to develop a new Medinilla variety with good growing habit and attractive inflorescence.

The New Medinilla ‘ROYAL PEARLZ’ originated from a cross made in the controlled breeding progeam by the inventors in 2008, in Assendelft, The Netherlands. The female or seed parent is the Medinilla selection designated ‘834918227’ (unpatented). ‘ROYAL PEARLZ’ was discovered and selected by the inventors in 2010, as a single flowering plant within the progeny of the stated cross in a controlled environment in Assendelft, The Netherlands.

Asexual reproduction of the new Medinilla hybrid ‘ROYAL PEARLZ’ performed by vegetative means by cuttings was first performed in 2010, in Assendelft, The Netherlands. The first ‘ROYAL PEARLZ’ plants propagated through the use of the cuttings flowered in 2011, in Assendelft, The Netherlands, and have demonstrated that the combination of characteristics as herein disclosed for the new cultivar are firmly fixed and retained through successive generations of asexual reproduction. The new cultivar reproduces true-to-type.

### BRIEF DESCRIPTION OF THE INVENTION

The following traits have been repeatedly observed and are determined to be unique characteristics of ‘ROYAL PEARLZ’ which in combination distinguish this Medinilla hybrid as a new and distinct cultivar:
1. Solid growth habit.
2. Erect shrub plant, measuring about 30 cm in height (above the pot when first flowering);
3. Green colored leaves, measuring about 15-20 cm in length and about 10-20 cm in width;
4. Pendulous panicles with red-purple (RHS 72C) colored flowers;

### COMMERCIAL COMPARISON

Of the many commercial cultivars known to the present inventors, the most similar in comparison to the new Medinilla ‘ROYAL PEARLZ’ is the Medinilla ‘MAGNIFICA’. Plants of the new Medinilla ‘ROYAL PEARLZ’ differ from plants of Medinilla ‘MAGNIFICA’ primarily in leaf size and bract size. Plants of ‘ROYAL PEARLZ’ have smaller leaves and smaller sized bracts than plants of “MAGNIFICA’.

### BRIEF DESCRIPTION OF THE PHOTOGRAPHS

The accompanying photograph illustrates the overall appearance of the new Medinilla ‘ROYAL PEARLZ’ showing the colors as true as is reasonably possible with colored reproductions of this type. Colors in the photograph may differ slightly from the color values cited in the detailed botanical description which accurately describes the color of ‘ROYAL PEARLZ’.

FIG. 1 shows a side view perspective of a typical potted flowering plant of ‘ROYAL PEARLZ’ at about 50 weeks from potting.

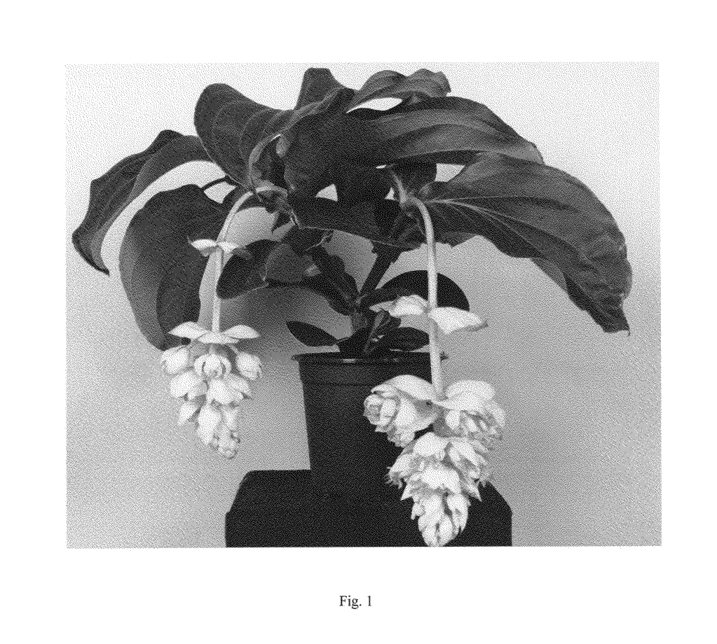

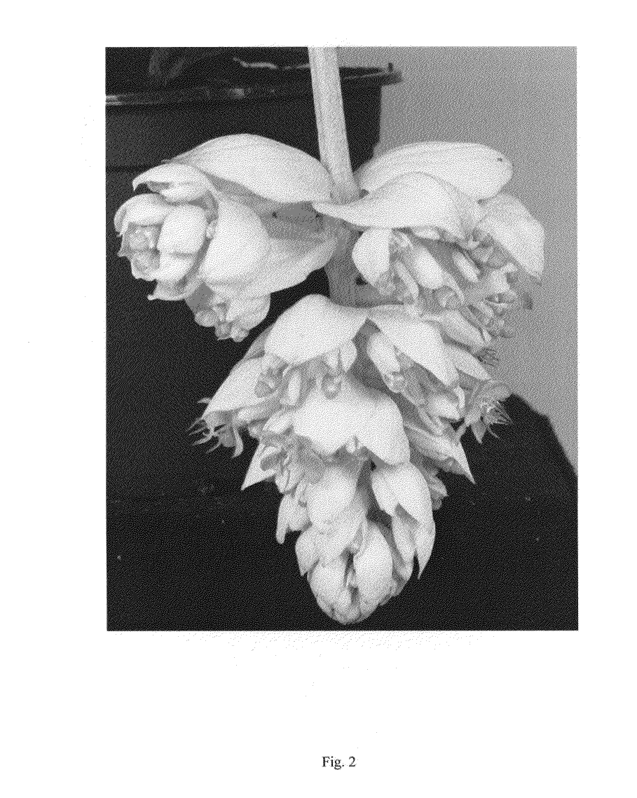

### DETAILED BOTANICAL DESCRIPTION

‘ROYAL PEARLZ’ has not been observed under all possible environmental conditions. The phenotype of the new cultivar may vary with variations in environment such as temperature, light intensity, day length, fertilizers and humidity without any change in the genotype of the plant.

The aforementioned photograph, together with the following observations, measurements and values describe the new Medinilla “ROYAL PEARLZ’ as grown in a greenhouse in Assendelft, The Netherlands, under conditions which closely approximate those generally used in commercial practice.

Color references are made to The Royal Horticultural Society Colour Chart (R.H.S.), 2001 Edition, except where general colors of ordinary significance are used. Color values were taken under daylight conditions in a greenhouse in Assendelft, The Netherlands. The age of the plants of ‘ROYAL PEARLZ’ described is about 50 weeks from potting.
Classification:
   Botanical.—Medinilla sp.
Parentage:
   Female parent.—Medinilla selection designated ‘834918227’ (unpatented).
   Male parent.—Medinilla selection designated ‘834917015’ (unpatented).
Propagation: Cuttings
Plant:
   General appearance and form.—Height: About 30 cm Diameter: About 45-70 cm Shape: Erect shrub.
   Growth habit.—Stem.
   Plant vigor.—Good.
   Flowering season.—From April till October.
   Cold tolerance.—Frost tender, temperatures below 5 C may damage plants.
   Fragrance.—None.
Foliage:
   Quantity.—About 18.
   Size.—Length: About 15-20 cm Width: About 10-20 cm.
   Overall shape.—elliptical.
   Apex shape.—Cuspidate.
   Base shape.—Acute.
   Margin.—Slightly Undulate.
   Texture.—smooth.
   Color.—Leaf color can vary somewhat depending on growing conditions Upper Surface: Glossy green, ranges between RHS 137A and RHS 139A Lower Surface: Green, closest to RHS 143C.
   Venation.—sunken.
Inflorescence:
   Shape.—Pendulous panicles.
   Amount.—about 4 (depending on the size of the plant).
   Size.—Length: About 36 cm (including peduncle) Width: About 15 cm.
   Color.—red-purple, closest to RHS 72C.
   Corolla.—Number: more than 150 per panicle.
   Petals.—Number: 5 per flower Length: 12 mm Width: 7 mm Overall Shape: obovate, asymmetrical Apex Shape: obtuse Color: Upper Surface: red-purple, closest to RHS 72C Lower Surface: red-purple, closest to RHS 72C Sepals: growth together, enclosing the ovary Color: red-purple, closest to RHS 65A Bracts: Quantity: 2-4 per crown Arrangement: verticillate Size: Length: about 1-5 cm Width: 7 mm-5.5 cm Overall Shape: elliptical.
Reproductive organs:
   Androecium.—Stamen: Number: 10 per flower Length: 10 mm Diameter: 1 mm Color: light yellow, with a white base (too small to qualify RHS value) Anther: Length: 8 mm Color: violet, closest to RHS 88B, with a yellow base (too small to qualify RHS value) Pollen: not visible Gynoecium: Pistil: Number: 1 per flower Length: 19 mm Stigma: Width: <1 mm Color: white, too small to qualify RHS value Style: Length: 16 mm Color: White, closest to RHS 155B Ovary: Shape: round Position: inferior Length: 4 mm Diameter: 4 mm Color: white closest to RHS 155C.
Seeds: No seeds produced
Fruits: No fruit produced
Disease/pest resistance: No observations made
Disease/pest susceptibility: No observations made

What is claimed is:

1. A new and distinct Medinilla plant named ‘ROYAL PEARLZ’, as illustrated and described herein.
